SEQUENCE LISTING (6 sequences)

SEQ ID NO: 1 - DNA, 231 bp, Escherichia coli
atggacaaggtgctgaaccgggaggaaagcctgcagctgatggacctgctgggcctggaaagaagcgcctggggcaacatccccctgatgcggaaggcctacctgaagaagtgcaaagagttccaccccgacaagggcggcgacgaggaaaagatgaagaagatgaacaccctgtacaagaaaatggaagatggcgtgaagtacgcccatcagcccgacttcggcggcttc

SEQ ID NO: 2 - DNA, 72 bp, Simian virus 40
accaacgactgattaactctacgtacgaaacgtatgaagacggacgacccctcggacccctgaaaggtgtgg

SEQ ID NO: 3 - DNA, 1254 bp, Mus musculus
atgcgagccgttcctctgcccctgagccggacagcaagcctcagccttggcttcttgctcctgctttctctctgcctggacccaggccaagccaaggagttgaagtttgtgacattggtgtttcggcatggagaccgaggtcccatcgagacctttcctaccgaccccattacagaatcctcgtggccacaaggatttggccaactcacccagtggggcatggaacagcactacgaacttggaagttatataaggaaaagatacggaagattcttgaacgacacctataagcatgatcagatttatatccggagcacagatgtggacaggactttgatgagtgctatgacaaaccttgcagccctgtttcctccagaggggatcagcatctggaatcctagactgctctggcagcccatcccagtgcacaccgtgtctctctctgaggatcggttgctgtacctgcctttcagagactgccctcgttttgaagaactcaagagtgagactttagaatctgaggaattcttgaagaggcttcatccatataaaagcttcctggacaccttgtcgtcgctgtcgggattcgatgaccaggatctttttggaatctggagtaaagtttatgaccctttattctgcgagagtgttcacaatttcaccttgccctcctgggccaccgaggacgccatgattaagttgaaagagctatcagaattatctctgctatcactttatggaattcacaagcagaaagagaaatctcgactccaagggggcgtcctggtcaatgaaatcctcaagaatatgaagcttgcaactcagccacagaagtataaaaagctggtcatgtattccgcacacgacactaccgtgagtggcctgcagatggcgctagatgtttataatggagttctgcctccctacgcttcttgccacatgatggaattgtaccatgataagggggggcactttgtggagatgtactatcggaatgagacccagaacgagccctacccactcacgctgccaggctgcacccacagctgccctctggagaagtttgcggagctactggacccggtgatctcccaggactgggccacggagtgtatggccacaagcagccaccaagtgctgagggttatccttgccactacattttgcctggtaaccgggatcctggtgatacttctgcttgtcctcatccgccatgggccctgctggcagagagatgtgtatcggaacatctga

SEQ ID NO: 4 - DNA, 1589 bp, Artificial
  mPAP A
atggacaaggtgctgaaccgggaggaaagcctgcagctgatggacctgctgggcctggaaagaagcgcctggggcaacatccccctgatgcggaaggcctacctgaagaagtgcaaagagttccaccccgacaagggcggcgacgaggaaaagatgaagaagatgaacaccctgtacaagaaaatggaagatggcgtgaagtacgcccatcagcccgacttcggcggcttcatgagagccgtgcccctgcccctgagcaggaccgccagcctgagcctgggcttcctgctgctgctgtctctgtgcctggaccctggacaggctaaagaactgaagttcgtgaccctggtgttccggcacggcgacagaggccccatcgagaccttccccaccgaccccatcaccgagagcagctggccccagggcttcggccagctgacccagtggggcatggaacagcactacgagctgggcagctacatccggaagagatacggccggttcctgaacgacacctacaagcacgaccagatctacatccggtctaccgacgtggaccggaccctgatgagcgccatgaccaacctggccgccctgttcccccccgagggcatcagcatctggaacccccggctgctgtggcagcccatccccgtgcacaccgtgagcctgagcgaggacaggctgctgtacctgcccttcagagactgcccccggttcgaggaactgaagagcgagaccctggaaagcgaagagttcctgaagcggctgcacccctacaagagctttctggacaccctgagcagcctgagcggcttcgacgaccaggacctgttcggcatctggtccaaggtgtacgaccccctgttctgcgagagcgtgcacaactttaccctgccctcctgggccaccgaggacgccatgattaagctgaaagagctgtccgagctgtctctgctgtccctgtacggcatccacaagcagaaagagaagagccggctgcagggcggcgtgctggtgaacgagatcctgaagaacatgaagctggccacccagccccagaagtacaagaaactggtcatgtacagcgcccacgacaccaccgtgagcggcctgcagatggccctggacgtgtacaacggcgtgctgcccccctacgccagctgccacatgatggaactgtaccacgataagggcggccacttcgtggagatgtactaccggaacgagacccagaacgagccctaccccctgaccctgcctggctgcacccacagctgccccctggaaaagttcgccgagctgctggaccccgtgatctcccaggactgggccacagagtgcatggccacctccagccaccaggtgctgcgggtgatcctggccaccaccttttgcctggtgaccggcatcctggtgattctgctgctggtgctgatccgccacggcccctgctggcagcgggacgtgtaccggaacatctaccaacgactgattaactctacgtacgaaacgtatgaagacggacgacccctcggacccctgaaaggtgtgggctacccctacgacgtgcccgactacgcctctag

SEQ ID NO: 5 - DNA, 1357 bp, Artificial
  mPAP B
atgagagccgtgcccctgcccctgagcaggaccgccagcctgagcctgggcttcctgctgctgctgtctctgtgcctggaccctggacaggctaaagaactgaagttcgtgaccctggtgttccggcacggcgacagaggccccatcgagaccttccccaccgaccccatcaccgagagcagctggccccagggcttcggccagctgacccagtggggcatggaacagcactacgagctgggcagctacatccggaagagatacggccggttcctgaacgacacctacaagcacgaccagatctacatccggtctaccgacgtgaccggaccctgatgagcgccatgaccaacctggccgccctgttcccccccgagggcatcagcatctggaacccccggctgctgtggcagcccatccccgtgcacaccgtgagcctgagcgaggacaggctgctgtacctgcccttcagagactgcccccggttcgaggaactgaagagcgagaccctggaaagcgaagagttcctgaagcggctgcacccctacaagagctttctggacaccctgagcagcctgagcggcttcgacgaccaggacctgttcggcatctggtccaaggtgtacgaccccctgttctgcgagagcgtgcacaactttaccctgccctcctgggccaccgaggacgccatgattaagctgaaagagctgtccgagctgtctctgctgtccctgtacggcatccacaagcagaaagagaagagccggctgcagggcggcgtgctggtgaacgagatcctgaagaacatgaagctggccacccagccccagaagtacaagaaactggtcatgtacagcgcccacgacaccaccgtgagcggcctgcagatggccctggacgtgtacaacggcgtgctgcccccctacgccagctgccacatgatggaactgtaccacgataagggcggccacttcgtggagatgtactaccggaacgagacccagaacgagccctaccccctgaccctgcctggctgcacccacagctgccccctggaaaagttcgccgagctgctggaccccgtgatctcccaggactgggccacagagtgcatggccacctccagccaccaggtgctgcgggtgatcctggccaccaccttttgcctggtgaccggcatcctggtgattctgctgctggtgctgatccgccacggcccctgctggcagcgggacgtgtaccggaacatctaccaacgactgattaactctacgtacgaaacgtatgaagacggacgacccctcggacccctgaaaggtgtgggctacccctacgacgtgcccgactacgcctctag

SEQ ID NO: 6 - DNA, 1496 bp, Artificial
  mPAP C
atggacaaggtgctgaaccgggaggaaagcctgcagctgatggacctgctgggcctggaaagaagcgcctggggcaacatccccctgatgcggaaggcctacctgaagaagtgcaaagagttccaccccgacaagggcggcgacgaggaaaagatgaagaagatgaacaccctgtacaagaaaatggaagatggcgtgaagtacgcccatcagcccgacttcggcggcttcaaagaactgaagttcgtgaccctggtgttccggcacggcgacagaggccccatcgagaccttccccaccgaccccatcaccgagagcagctggccccagggcttcggccagctgacccagtggggcatggaacagcactacgagctgggcagctacatccggaagagatacggccggttcctgaacgacacctacaagcacgaccagatctacatccggtctaccgacgtgggccggttcctgaacgacacctacaagcacgaccagatctacatccggtctaccgacgtgagcatctggaacccccggctgctgtggcagcccatccccgtgcacaccgtgagcctgagcgaggacaggctgctgtacctgcccttcagagactgcccccggttcgaggaactgaagagcgagaccctggaaagcgaagagttcctgaagcggctgcacccctacaagagctttctggacaccctgagcagcctgagcggcttcgacgaccaggacctgttcggcatctggtccaaggtgtacgaccccctgttctgcgagagcgtgcacaactttaccctgccctcctgggccaccgaggacgccatgattaagctgaaagagctgtccgagctgtctctgctgtccctgtacggcatccacaagcagaaagagaagagccggctgcagggcggcgtgctggtgaacgagatcctgaagaacatgaagctggccacccagccccagaagtacaagaaactggtcatgtacagcgcccacgacaccaccgtgagcggcctgcagatggccctggacgtgtacaacggcgtgctgcccccctacgccagctgccacatgatggaactgtaccacgataagggcggccacttcgtggagatgtactaccggaacgagacccagaacgagccctaccccctgaccctgcctggctgcacccacagctgccccctggaaaagttcgccgagctgctggaccccgtgatctcccaggactgggccacagagtgcatggccacctccagccaccaggtgctgcgggtgatcctggccaccaccttttgcctggtgaccggcatcctggtgattctgctgctggtgctgatccgccacggcccctgctggcagcgggacgtgtaccggaacatctaccaacgactgattaactctacgtacgaaacgtatgaagacggacgacccctcggacccctgaaaggtgtgggctacccctacgacgtgcccgactacgcctctag

(19) United States
(12) Patent Application Publication
(10) Pub. No.: US 2013/0115239 A1
(43) Pub. Date: May 9, 2013

(54) PROSTATE CANCER DNA VACCINE

(71) Applicants: Marcus Groettrup, Konstanz (DE); Peter Oehlschlaeger, Konstanz (DE)

(72) Inventors: Marcus Groettrup, Konstanz (DE); Peter Oehlschlaeger, Konstanz (DE)

(73) Assignee: UNIVERSITÄT KONSTANZ, Konstanz (DE)

(21) Appl. No.: 13/513,531

(22) PCT Filed: Nov. 25, 2010
(86) PCT No.: PCT/EP10/68209

§ 371 Date: Jan. 14, 2013

(30) Foreign Application Priority Data
Dec. 1, 2009 (GB) 0921088.1

(51) Int. Cl. A61K 48/00; A61K 38/46

(52) U.S. Cl. 424/192.1; 536/23.2; 435/320.1; 435/91.1; 435/325; 435/366; 435/354

(57) ABSTRACT

The present invention concerns an (adjuvant) treatment or prevention option for the treatment and prevention of prostate cancer. In particular, it pertains to the provision of recombinant, optimized PAP genes which are useful as DNA vaccines for the above treatment or prevention.

### SUMMARY

Treatment of prostate cancer (PCa) patients by surgery or radiotherapy may be effective for presumed organ-confined tumors—however, about one-third of men with PCa will have progressive or metastatic disease within 10 years after first diagnosis. A promising possibility to make a therapeutic treatment more effective could be the development of a DNA vaccine. The present invention has developed an artificial prostate-acid-phosphatase (PAP)-based DNA vaccine. To increase the efficacy features like Kozak-sequence, SV-40 enhancer as a nuclear signal and fusion of PAP to the J domain of SV40 large T to enhance cross-presentation have been combined.

### BACKGROUND ART

A. Prostate Cancer

PCa is the Second Leading Cause of Cancer-Related Deaths among Men in the USA.

In the United States approximately 29.000 men died due to PCa in 2003 [Jemal, A, et al., CA Cancer J Clin 53 (2003) 5-26]. In Germany, nearly 50.000 new PCa cases per annum were detected and PCa represents with 22.3% the most common localization of malignant tumors in men [http://www.ekr.med.uni-erlangen.de/GEKID/Doc/kid2006.pdf]. The risk factors are essentially still unclear—probably, obesity, a diet rich in fat and calories, a lack of movement and smoking are all more or less associated with the development of PCa. Recently, a previously unknown virus (XMRV, related to murine leukaemia virus) has been brought in connection with prostate cancer [American Society for Clinical Oncology—Prostate Cancer Symposium, San Francisco, Calif., USA, Feb 24-26, 2006].

Surgery, chemotherapy and/or radiotherapy are the standard therapies for local tumor treatment. Nevertheless, about one-third of PCa patients will develop progressive or metastatic disease within 10 years after first diagnosis [Oefelein, M. G. et al., J Urol 158 (1997) 1460-1465]. In early metastatic disease androgen ablation is effective, but in most cases androgen-independent tumors will develop., Frequently, no effective treatment for androgen-independent disease is subsequently available.

A promising possibility to render a therapeutic treatment more effective could be the development of a prostate-specific vaccine.

DNA Vaccines

The development of a Therapeutic Vaccine against PCa is a Promising Approach for a(n Adjuvant) Therapy

Vaccine-based strategies are excellent treatment options to eradicate micrometastatic disease [McNeel, D. G. et al., Immunol Lett 96 (2005) 3-9 and McNeel, D. G. et al., Cancer Chemother Biol Response Modif 22 (2005) 247-261].

As discussed above, conventional means like surgical intervention or other conventional therapies are so far usually insufficient in cases of metastasing prostate cancer in general and the androgen independent form thereof in particular. A vaccine-based strategy would activate the patient's own immune system, which could then recognise all metastases and single metastatic cells and eliminate them in an optimal case, should however at least decrease the growth of the cancer. This therapy/prevention of further growth, could stand alone or could be an adjuvant therapy, whereby “adjuvant” in the present sense means to support the above conventional therapy options.

In DNA vaccination, a DNA—derived from a self-antigen present on tumour cells—is administered into the muscle. If properly designed, it will work to activate the already present cytotoxic T-lymphocytes circulating in the patient's blood. These cytotoxic T-lymphocytes, directed against self-antigens, are those remaining in a subject, even after the (necessary) elimination of most of such cytotoxic T-lymphocytes during embryogenesis.

These remaining T-lymphocytes are usually characterised by a binding constant to the self-antigens in the low-affinity range; this low-affinity binding on the one hand enabled them to survive embryogenesis but prevents them from effectively clearing cells from the body that present the above self-antigens.

This situation is a challenge in current research programmes which—although having perhaps even identified suitable target self-antigens on (potential) tumour cells—usually cannot achieve a sufficient level of activation of the CTLs (i.e. cytotoxic T-lymphocytes) already present in the body and directed against these self-antigens.

it was suggested that the doubling time of serum PSA (prostate specific antigen) in stage D0 PCa (patients after therapy and with increasing PSA serum level) is associated with the time to the detection of metastases and death from PCa [Freedland, S. J. et al., JAMA 294 (2005) 433-439]. As a consequence, patients in D0 PCa stage are part of a population at high risk of developing micrometastatic disease and should particularly benefit from adjuvant vaccine therapy.

DNA-Based Therapeutic Vaccinations are Safe and Could be a Therapy Per Se or Serve as an Ideal Supplement to Existing Therapies

As compared to protein- or peptide-based vaccines a DNA vaccine has remarkable advantages. For example, its production costs are relatively low and predictable. DNA is stable and does not require refrigeration for storage. There are no unwanted immune reactions against other components of the vaccine as e.g. those observed in case of vector based-vaccines; thus, DNA vaccines can be used for repeated boosting [Liu, M. A., Nat Med 4 (1998) 515]. Clinical studies in humans demonstrated the absence of severe side effects after DNA immunization.

In the field, formerly the concern was voiced that integration of plasmid DNA could lead to an induction of oncogenes or inactivation of tumor suppressor genes. However, it was shown in mouse experiments that even under the most unfavorable conditions the mutation rate is not detectable, i.e. at least 3000 times below the frequency of spontaneous mutations [Martin, T. et al., Hum Gene Ther 10 (1999) 759-768 and Nichols, W. W. et al., Ann N Y Acad Sci 772 (1995) 30-39].

In clinical trials, mostly HIV genes are currently tested in that regard and a complete lack of severe side effects was published [MacGregor, R. R. et al., The J of Inf Dis 178 (1998) 92-100]. A presence of DNA-specific antibodies was not reported. In contrast, a humoral immune response after DNA immunization was found in a mouse model [Mor, G. et al., Hum Gene Ther 8 (1997) 293-300]. The number of anti-DNA IgG secreting B cells increased two- to three-fold shortly after vaccination but no symptoms of autoimmunity were detected [Katsumi, A. et al., Hum Gene Ther 5 (1994) 1335-1339 and Mor, G. et al., Hum Gene Ther 8 (1997) 293-300 and Xiang, Z. Q. et al., Virol 209 (1995) 569-579 and Gilkeson, G. S. et al., J Immunol 161 (1998) 3890-3895].

Specific Immune Therapy Depends on a Target Antigen That is Ideally Expressed Exclusively in Tumor Tissue

The current immunotherapies of PCa are hampered by the lack of validated tumor antigens, although different potential prostate antigens have been identified [Tricoli, J. V. et al., Clin Cancer Res 10 (2004) 3943-3953]. Tumor antigens used for therapeutic vaccination have to fulfill at least two essential criteria. First, the antigen should be restricted to non-vital organs (here: prostate tissue). Second, the antigen should be expressed on target cells in a sufficient amount in order to provide cytotoxic efficiency. Indeed, it was shown, that the induction of an immune response against the self-antigen PSA is possible [Wei, C. et al., Proc Natl Acad Sci 94 (1997) 6369-6374]. It has also been shown, in rats, that the immunological tolerance can be broken by immunization with a non-optimized DNA vaccine encoding rat PAP as shown by measurement of the immune response against PAP.

In this situation it would be highly desirable to have an additional, effective therapy or adjuvant therapy to substitute or replace the conventional therapies.

It is thus an object of the present invention to provide such a therapy and/or prevention

In order to enhance expression, codons were optimized for the human system (which is nearly identical to the murine system). Moreover, during the optimization process different cis-acting sequences (internal TATA-boxes, chi-sites and ribosomal entry sites, AT-rich or GC-rich (>80% or <30%) sequence stretches, ARE, INS, CRS sequence elements, repeat sequences and RNA secondary structures, (cryptic) splice donor and acceptor sites, branch points) were avoided.

### DESCRIPTION OF THE PRESENT INVENTION

In this invention, the inventors have focused on prostate specific antigen (PAP) as target for the development of therapeutic DNA vaccine. The PAP antigen is highly expressed in prostate tissue [Cunha, A. C. et al., Cancer Letters (2005) 1-10] but not in any other tissues investigated [Sinha, A. A. et al., Anticancer Res 18 (1998) 1385-1392 and Solin, T. et al., Biochim Biophys Acta 1048 (1990) 72-77] and is expressed in rodents as well as in humans; hence, the present inventors determined this antigen to be of outstanding interest for preclinical testing. On the other hand, PAP is a secreted molecule—in general cell surface and intracellular molecules are thought to represent the best tumor targets. For this reason, a signal-peptide deleted PAP-antigen has also been generated.

The present invention thus focuses on the following aspects:

1. A preventive or therapeutic agent for the prevention or treatment of prostate cancer, wherein said agent comprises a recombinant Prostate Acid Phosphatase (PAP) nucleic acid or a functional equivalent thereof.

2. The preventive or therapeutic agent according to item 1, wherein the functional equivalent shows a homology of at least 70, preferably 80, even more preferably 90% to mouse PAP DNA, as represented by SEQ ID No: 3.

3. The preventive or therapeutic agent according to items 1 or 2, wherein said agent is a recombinant DNA and a functional equivalent, wherein said functional equivalent comprises an epitope or a minigene of a PAP nucleic acid.

4. The preventive or therapeutic agent according to any one of items 1 to 3, in the form of a fusion polynucleotide comprising
deletion of the signal sequence,
codon optimization for humans,
linkage with an SV 40 enhancer,
linkage with a J-domain and/or
linkage with a Kozak sequence.

5. The preventive or therapeutic agent according to any of items 1 to 4, wherein said agent is selected from the group consisting of mPAP A, mPAP B and/or mPAP C.

6. Use of a recombinant Prostate Acid Phosphatase (PAP) nucleic acid or a functional equivalent thereof for the prevention or treatment of prostate cancer.

7. Use of a preventive or therapeutic agent according to item 6 wherein the functional equivalent shows a homology of at least 70, preferably 80, even more preferably 90% to mouse PAP DNA, as represented by SEQ ID No: 3.

8. Use of a preventive or therapeutic agent according to item 6 or 7, wherein said agent is a recombinant DNA, and a functional equivalent, wherein said functional equivalent comprises an epitope or a minigene of a PAP nucleic acid.

9. Use according to any one of items 6 to 8 in the form of a fusion polynucleotide, comprising
a deletion of signal sequence,
codon optimization for humans,
linkage with an SV 40 enhancer,
linkage with a J-domain and/or
linkage with a Kozak sequence.

10. Use of a preventive or therapeutic agent according to any of items 6 to 9, wherein said agent is selected from the group consisting of mPAP A, mPAP B and/or mPAP C.

11. Use according to any one of items 6 to 10, wherein the treatment or prevention of prostate cancer is accompanied by or follows a treatment with further conventional therapy.

12. Vector, comprising the nucleic acid as defined in any one of items 1 to 5.

13. Host cell, comprising the vector according to item 12.

14. Method for the production of a nucleic acid as defined in item 4 or 5, comprising the following steps:
a) providing a recombinant DNA comprising a PAP DNA or a functional equivalent thereof, wherein at least all introns have been deleted and/or
b) deleting the signal sequence and/or
c) codon-optimizing the resultant recombinant DNA and/or
d) linking the PAP DNA or functional equivalent with an SV enhancer, and/or with a J-domain and/or with a Kozak sequence, and
e) expressing the resultant construct.

15. Method according to item 14, wherein all of steps a) to e) are carried out.

Definitions

PAP: Prostate Acid Phosphatase, a prostate specific antigen, is an enzyme produced by the prostate. It may be found in increased amounts in men who have prostate cancer; reference to PAP here is meant to include all possible variants and functional equivalents thereof which share the function of PAP.

mPAP: is the basic gene used herein exemplary for the design of the desired constructs. It is derived from the mouse PAP, without introns and signal sequence and has, exemplary, the sequence as given in SEQ ID No: 3.

Functional equivalent: a functional equivalent—used here interchangeably with “variant”—of a Prostate Acid Phosphatase is herein any equivalent thereof which still has the present desired function, namely effectiveness as a DNA vaccine for prostate cancer. Whether or not an equivalent is indeed functional in the present sense can be determined by carrying out e.g. the C1 Tumor Regression Experiments as described in the 4^{th} Experiment of the present application. Functional equivalents which have at least 50% effectiveness as compared to the data shown for PAP C in the 4^{th} Experiment are considered to be “functional equivalent”. In preferred embodiments, these functional equivalents have at least 60%, even more preferred 70%, further preferred 80%, and particularly preferred 90% effectiveness compared to the results of the 4^{th} Experiment described here; as an example, a ‘functional equivalent’ can be a DNA fragment of PAP consisting of or comprising an epitope and/or a DNA consisting of several epitopes, linked together to form a so-called “minigene”. The expression minigene is well known to a person skilled in the art and depicts nucleic acid fragments, which have been engineered to comprise or consist of two or more epitopes.

Nucleic acid: in the present context nucleic acid encompasses all nucleic acids and fragments thereof as known to a person skilled in the art. That is, the nucleic acid according to the present invention can be a DNA or RNA; if it is RNA, it can be an mRNA and siRNA. In the case of a DNA it can be a cDNA. All possible fragments of nucleic acids are also encompassed. “Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g. degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994). The term nucleic acid is used interchangeably with gene, cDNA, mRNA, oligonucleotide and polynucleotide.

Conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

Homology: “homology” in the present context is used interchangeably with “similarity” or “identity”. As used herein, two sequences are “homologous” or “similar” to each other where they have at least 85% sequence similarity to each other when aligned using either the Needleman-Wunsch algorithm or the “BLAST 2 sequences” algorithm described by Tatusova & Madden, 1999, FEMS Microbiol Leff. 174:247-250. Where amino acid sequences are aligned using the “BLAST 2 sequences algorithm,” the Blosum 62 matrix is the default matrix.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SOS at 65° C., followed by one or more washes at 0.2×SSC, 1% SDS at 65° C.

Vaccine: A vaccine is a biological preparation (here: DNA encoding for PAP) that improves immunity to a particular disease (here: prostate cancer).

Therapeutic DNA vaccination: A technique for inducing an immune response against an already existing disease (here cancer) by injecting genetically engineered DNA.

Cross-presentation: The ability of certain antigen-presenting cells to take up, process and present extracellular antigens with MHC class I molecules to CD8⁺ T cells (cytotoxic T cells).

Cross-priming: Cross-priming describes the stimulation of naive cytotoxic CD8⁺ T cells by cross-presentation.

Hsp73 binding DnaJ-like domain: The heat shock protein 73 (hsp73) is abundantly and constitutively expressed in the cytosol of mammalian cells and can facilitate protein degradation in a novel TAP-independent lysosomal degradation pathway. The “DnaJ-like domain” is originally derived from the prokaryotic heat shock protein DnaJ and binds to mammalian hsp73. Its sequence is well known in the field. Exemplary and as used herein, the DnaJ-like domain (or “J-domain”) has a sequence as shown in SEQ ID NO: 1. The PAP fused to the DnaJ-like domain should optimally in consequence enter the novel lysosomal pathway of surrounding dendritic cells after cell death of the expressing cell.

Large T antigen: Antigen of Polyoma-viruses (e.g. Simian virus type 40) which plays a key role in regulating the viral life cycle by binding to the viral origin of DNA replication where it promotes DNA synthesis. As the polyomavirus relies on the host cell machinery to replicate the host cell needs to be in s-phase for starting replication. Due to this, large T-antigen also modulates cellular signaling pathways to stimulate progression of the cell cycle by binding to a number of cellular control proteins. This is achieved e.g. by a two pronged attack of inhibiting tumor suppressing genes p53 and members of the retinoblastoma (pRB) family, and stimulating cell growth pathways by binding cellular DNA and ATPase-helicase. This abnormal stimulation of the cell cycle is a powerful force for oncogenic transformation. The SV40 antigen is well known to a person skilled in the art. Exemplary, and as used herein, the SV40 sequence is as depicted in SEQ ID NO: 2.

HPV16 E7: The main oncogene/oncoprotein of the Human Papillomavirus Type 16 inducing transformation in HPV-16 E7 transfected cells.

Kozak sequence: Again, the Kozak sequence is well-known to a person skilled in the art.

Codon-optimization: A strategy in which codons within a gene are changed by in vitro mutagenesis to the preferred codons, without changing the amino acids of the synthesized protein leading to enhanced expression of the encoded protein. This strategy is well-known to a skilled person and can be carried out with respective software programmes, or by specialized firms.

RMA-S: A cell line origin of BL/6 mice, unable to load epitopes to empty MHC I molecules at the endoplasmatic reticulum. Empty MHC I molecules onto the cell surface will accept external epitopes for binding.

pPOE: plasmid “Peter Oehlschlaeger”, immunization vector driven by an CMV-promotor, kanamycin selectable providing highly optimized CpG motifs in the backbone.

BL/6: Mouse strain commonly used in immunology.

TRAMP: Transgenic Adenocarcinoma of Mouse Prostate, mouse model with BL/6 background developing prostatic intraepithelial neoplasia that will become invasive and metastasize primarily to the lymph nodes and lungs.

Conventional Therapy: in the present context this is a conventional therapy of prostate cancer and examples thereof are surgical intervention and/or radiotherapy and/or chemotherapy, with examples of surgery being
Pelvic lymphadenectomy: A surgical procedure to remove the lymph nodes in the pelvis. A pathologist views the tissue under a microscope to look for cancer cells. If the lymph nodes contain cancer, the doctor will not remove the prostate and may recommend other treatment.
Radical prostatectomy: A surgical procedure to remove the prostate, surrounding tissue, and seminal vesicles. There are 2 types of radical prostatectomy:
Retropubic prostatectomy: A surgical procedure to remove the prostate through an incision (cut) in the abdominal wall. Removal of nearby lymph nodes may be done at the same time.
Perineal prostatectomy: A surgical procedure to remove the prostate through an incision (cut) made in the perineum (area between the scrotum and anus). Nearby lymph nodes may also be removed through a separate incision in the abdomen.

Radiation therapy: is a cancer treatment that uses high-energy x-rays or other types of radiation to kill cancer cells or keep them from growing. There are two types of radiation therapy. External radiation therapy uses a machine outside the body to send radiation toward the cancer. Internal radiation therapy uses a radioactive substance sealed in needles, seeds, wires, or catheters that are placed directly into or near the cancer. The way the radiation therapy is given depends on the type and stage of the cancer being treated.

There is an increased risk of bladder cancer and/or rectal cancer in men treated with radiation therapy.

Impotence and urinary problems may occur in men treated with radiation therapy.

Hormone therapy: is a cancer treatment that removes hormones or blocks their action and stops cancer cells from growing. Hormones are substances produced by glands in the body and circulated in the bloodstream. In prostate cancer, male sex hormones can cause prostate cancer to grow. Drugs, surgery, or other hormones are used to reduce the production of male hormones or block them from working.

Hormone therapy used in the treatment of prostate cancer may include the following:
Luteinizing hormone-releasing hormone agonists can prevent the testicles from producing testosterone. Examples are leuprolide, goserelin, and buserelin.
Antiandrogens can block the action of androgens (hormones that promote male sex characteristics). Two examples are flutamide and nilutamide.
Drugs that can prevent the adrenal glands from making androgens include ketoconazole and aminoglutethimide.
Orchiectomy is a surgical procedure to remove one or both testicles, the main source of male hormones, to decrease hormone production.
Estrogens (hormones that promote female sex characteristics) can prevent the testicles from producing testosterone. However, estrogens are seldom used today in the treatment of prostate cancer because of the risk of serious side effects.

Chemotherapy: is a cancer treatment that uses drugs to stop the growth of cancer cells, either by killing the cells or by stopping them from dividing. When chemotherapy is taken orally or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

High-intensity focused ultrasound: is a treatment that uses ultrasound (high-energy sound waves) to destroy cancer cells. To treat prostate cancer, an endorectal probe is used to make the sound waves.

Linked: in the present context means the provision of a linkage between two parts of a nucleic acid strand; preferred is a direct linkage, where the two linked parts do not have any intermittent molecules; in the present case, the constructs mPAP A, mPAP B and mPAP C were construed via “direct linkage” between their different components, by synthezing the designed nucleic acid strand in a linear fashion.

### EXAMPLES

Generation of the Artificial PAP Gene and Immunization Experiments

Different features have been investigated in order to study possible enhancement of the immunogenicity of the PAP-genes:
a) Codon optimization for optimal use in mammalian cells has not only proven beneficial for protein expression (e.g. in the case of EGFP, i.e. enhanced green fluorescing protein) but was recently also shown to increase the immunogenicity after DNA immunization [Liu, W. J. et al., Virology 301 (1) (2002) 43-52, Cid-Arregui, A. et al., J of Viral 77 (2003) 4928-4937 and Steinberg, T., Öhlschläger, P. et al., Vaccine 23 (9) (2005) 1149-1157].
b) In order to achieve more effective MHC-I cross-presentation and cross-priming of CTLs, we have placed the hsp73 binding DnaJ-like domain (“J-domain”) of the large T antigen (5′ATGGACAAGGTGCTGAACCGGGAGGAAAGCCTGCAGCTGATGGAC CTGCTGGGCCTGGAAAGAAGCGCCTGGGGCAACATCCCCCTGATGC GGAAGGCCTACCTGAAGAAGTGCAAAGAGTTCCACCCCGACAAGGGC GGCGACGAGGAAAAGATGAAGAAGATGAACACCCTGTACAAGAAAAT GGAAGATGGCGTGAAGTACGCCCATCAGCCCGACTTCGGCGGCTTC 3′=SEQ ID No: 1) directly in front (5′) of the therapeutic genes, see mPAP A and mPAP C, as described above, resulting in an hsp73 associated recombinant DNA vaccine. Thereby, hsp73-bound endogenous antigen is submitted to processing for MHC-I presentation, which facilitates cross-priming.
c) To facilitate the nuclear entry of the plasmid vector, we have taken advantage of a nuclear targeting sequence. The reasoning behind this is that only a minor part of the injected DNA is able to reach the nucleus where mRNA as a precursor of proteins is made. One of the major hurdles hereby is targeting through the nucleus membrane. The SV40 enhancer (5′ CCAACGACTGATTAACTCTACGTACGAAACGTATGAAGACGGACGAC CCCTCGGACCCCTGAAAGGTGTGG 3′, SEQ ID No: 2), contains binding sites for different ubiquitously expressed transcription factors (e.g. AP1, AP2, AP3, NF-κB) [Wildeman, A. G. et al., Biochem Cell Biol 66 (1988) 567-577], which offer a nuclear targeting sequence. It was hypothesized that the DNA-protein complex, consisting of (e.g. the above SV40-DNA and the bound transcription factor, leads to an increase in nuclear import. In the present case the inventors provided constructs, wherein the SV40 enhancer was linked directly to the 3′ end of the respective PAP gene. This linkage was carried out for all versions mPAP A, mPAP B and mPAP C, respectively.
d) A Kozak sequence (5′ GCCACC 3′) [Kozak, M., Nucleic Acids Res 20 (1987) 8125-8148] was introduced directly in front of the J-domain in the case of mPAP A, mPAP C and 5′ of the therapeutic gene in case of mPAP B;). It is defined as a consensus sequence which is located close to the start codon which increases the efficiency of initiation of translation.

The following artificial PAP genes were generated:

1. Therapeutic Gene as Basis:

We have used the murine PAP (“mPAP”) nucleotide sequence, without introns, but with the signal sequence, (5′ ATGCGAGCCGTTCCTCTGCCCCTGAGCCGGACAGCAAGCCTCAGCCTTG GCTTCTTGCTCCTGCTTTCTCTCTGCCTGGACCCAGGCCAAGCCAAGGA GTTGAAGTTTGTGACATTGGTGTTTCGGCATGGAGACCGAGGTCCCATC GAGACCTTTCCTACCGACCCCATTACAGAATCCTCGTGGCCACAAGGATT TGGCCAACTCACCCAGTGGGGCATGGAACAGCACTACGAACTTGGAAGT TATATAAGGAAAAGATACGGAAGATTCTTGAACGACACCTATAAGCATGAT CAGATTTATATCCGGAGCACAGATGTGGACAGGACTTTGATGAGTGCTAT GACAAACCTTGCAGCCCTGTTTCCTCCAGAGGGGATCAGCATCTGGAAT CCTAGACTGCTCTGGCAGCCCATCCCAGTGCACACCGTGTCTCTCTCTG AGGATCGGTTGCTGTACCTGCCTTTCAGAGACTGCCCTCGTTTTGAAGAA CTCAAGAGTGAGACTTTAGAATCTGAGGAATTCTTGAAGAGGCTTCATCC ATATAAAAGCTTCCTGGACACCTTGTCGTCGCTGTCGGGATTCGATGACC AGGATCTTTTTGGAATCTGGAGTAAAGTTTATGACCCTTTATTCTGCGAGA GTGTTCACAATTTCACCTTGCCCTCCTGGGCCACCGAGGACGCCATGATT AAGTTGAAAGAGCTATCAGAATTATCTCTGCTATCACTTTATGGAATTCAC AAGCAGAAAGAGAAATCTCGACTCCAAGGGGGCGTCCTGGTCAATGAAA TCCTCAAGAATATGAAGCTTGCAACTCAGCCACAGAAGTATAAAAAGCTG GTCATGTATTCCGCACACGACACTACCGTGAGTGGCCTGCAGATGGCGC TAGATGTTTATAATGGAGTTCTGCCTCCCTACGCTTCTTGCCACATGATG GAATTGTACCATGATAAGGGGGGGCACTTTGTGGAGATGTACTATCGGAA TGAGACCCAGAACGAGCCCTACCCACTCACGCTGCCAGGCTGCACCCAC AGCTGCCCTCTGGAGAAGTTTGCGGAGCTACTGGACCCGGTGATCTCCC AGGACTGGGCCACGGAGTGTATGGCCACAAGCAGCCACCAAGTGCTGA GGGTTATCCTTGCCACTACATTTTGCCTGGTAACCGGGATCCTGGTGATA CTTCTGCTTGTCCTCATCCGCCATGGGCCCTGCTGGCAGAGAGATGTGT ATCGGAACATCTGA=SEQ ID No: 3) which is about 80% identical to the human one.

2. Codon Optimization

The basic mPAP gene (“therapeutic gene” was then codon-optimized for the human system, which is nearly identical to the murine system [http://www.kazusa.or.jp/codon/index.html].

In order to enhance expression, codons were optimized for the human system (which is nearly identical to the murine system). Moreover, during the optimization process different cis-acting sequences (internal TATA-boxes, chi-sites and ribosomal entry sites, AT-rich or GC-rich (>80% or <30%) sequence stretches, ARE, INS, CRS sequence elements, repeat sequences and RNA secondary structures, (cryptic) splice donor and acceptor sites, branch points) were avoided.

3. Further Optimization
a) for the mPAP A version (SEQ ID NO: 4)

The J-domain, as described above, was directly linked 5′ to the basic mPAP gene, 3′ of the signal sequence, as described in step 1) above. The Kozak sequence, as described above was directly linked 5′ to the signal sequence, while the SV40 enhancer was directly linked 3′ to the basic mPAP gene. This version still has the signal sequence
b) for the mPAP B version (SEQ ID NO: 5)
as for a) above, though without the J-domain, resulting in a strand consisting of (from 5′ to 3′): Kozak sequence signal sequence—basic mPAP gene—SV40 enhancer. This version still has the signal sequence.
c) for the mPAP C version (SEQ ID NO: 6)
as for a) above, though without the signal sequence, resulting in a strand consisting of (from 5′ to 3′):
   Kozak sequence—J-domain—therapeutic mPAP gene—SV40 enhancer. This version has no signal sequence.

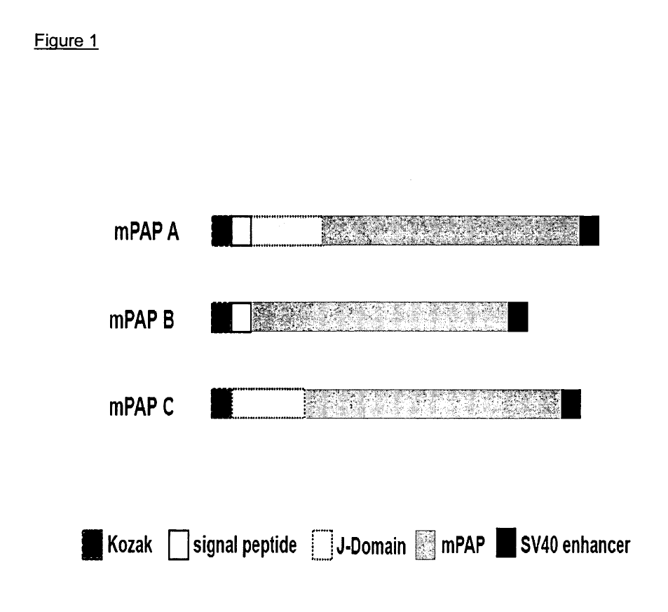

The three resulting nucleic acid strands
mPAP A
mPAP B, and
mPAP C
are graphically depicted in FIG. 1.

4. Addition of Detection Marker

An HA-tag was fused directly 3′ after the PAP gene in order to allow easy detection of expressed PAP-proteins via an HA-tag-specific first antibody, namely monoclonal mouse anti HA (mouse IgG1 isotype) (clone HA-7), Sigma, Deisenhofen (1: 10000 in PBS-Tween) and a secondary antibody (polyclonal goat anti-mouse Ig/HRP antibody (clone PO447), Dako, Germany GmbH, Hamburg, (1:1000 in PBS-Tween).

The HA-tag was included merely for detection; it should not contribute to the desired function of an enhancement of immunogenicity of the mPAP gene.

Although it has recently become clear in the field that several elements exist which might—in certain cases—enhance the antigenicity of a desired antigen, the underlying mechanism has still not been entirely elucidated and it is uncertain, whether or not specific elements will enhance specifically selected genes in the selected environment. Also, it appears that not all target genes can in fact be “activated” by such elements and if—and how—those elements will work out, if used in combination.

For example, there is a possibility that the activation of a desired target gene does not occur entirely (or even principally) via cytotoxic T-lymphocytes in all cases. There is evidence that specific antibodies are involved as well, acting here quite uncommonly in a context not properly understood.

Thus, for this reason alone, it is unpredictable, how or if a selected antigen can be activated and whether a specific combination of elements would be possible to allow a particularly advantageous level of activation.

After having selected PAP as a promising springboard, the present inventors were, however, able to show that a specific combination of several different approaches and elements indeed led to highly desirable antigen activation.

This specific combination of elements and features is the following:
a) use of PAP as therapeutic gene,
b) linkage with SV 40 enhancer,
c) linkage with J-domain,
d) codon optimization for humans
e) linkage with Kozak sequence, and/or
f) deletion of signal sequence.

In particular, in a preferred embodiment of the present invention, the mPAP gene used as basic therapeutic gene did not comprise any introns and no signal sequence and was codon-optimized for humans.

In a more preferred embodiment, the above construct additionally comprises a linkage to the J-domain, even more preferred additionally a linkage to an SV40 enhancer, further preferred additionally a linkage to the Kozak sequence.

Particularly preferred, the construct is as depicted in FIG. 1, in mPAP C.

### Experiments

1^{st} Experiment: Soft-Agar-Transformation-Assays

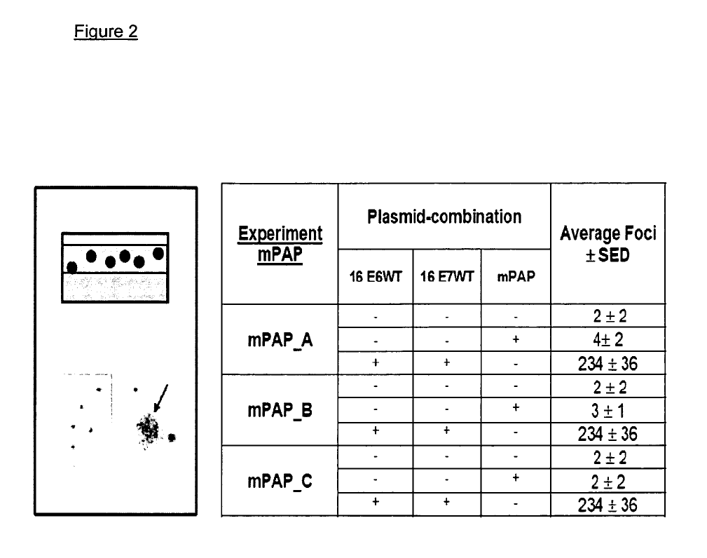

Murine fibroblasts normally need an attachment to the petri dish to grow (they are growing “anchorage dependent”), Transformation of these cells enables them to grow anchorage independent. For this assay, murine fibroblasts were transfected with the mPAP genes A, B and C as described above and seeded onto a so called “baselayer”, namely a layer of hardened soft agar, which prevents contact with the petri dish (FIG. 2, left box).

After four weeks untransformed cells were not able to grow (FIG. 2, left box, below left) whereas with HPV-16 oncogenes transformed cells proliferated resulting in the formation of so called “foci” (highlighted with the arrow). HPV-16 is an established positive control for this assay system. The combination of the oncogenes HPV-16 E6 and E7 wildtype were thus used as a positive control. The outcome of the experiment is, that all three artificial PAP genes tested were not transforming and therefore safe for use in humans.

2^{nd} Experiment: Elispot-Assays

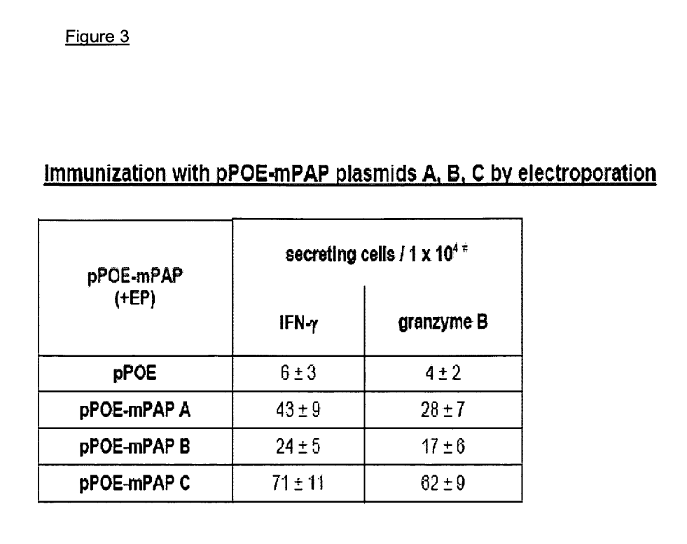

We have immunized BL/6 mice intramuscularly with the three different PAP-genes inserted into the pPOE plasmid, with techniques known to a person skilled in the art as in experiment 1 using conventional electroporation (EP) technology. One of the major hurdles for the DNA on its way to the nucleus is the cytoplasma membrane. EP mediates electrical fields, resulting in a transient increase in membrane permeability in cells of the target tissue. It is well known that EP-technology leads to an increased cellular immune response as measured by enhanced IFN-gamma and granzyme B secretion as typical markers of activated cytotoxic T lymphocytes. The shown data are based on “Elispot-Assays”, which detect secreted IFN-gamma respective granzyme B molecules of immune cells (FIG. 3). Empty pPOE was used as negative control. The data clearly demonstrate, that the mPAP C-gene is most immunogenic regarding the induction of cytotoxic T-lymphocytes.

Although the Elispot Assay shows that cytotoxic T-lymphocytes are induced (i.e. activated) it does not show whether they then actually kill target cells, which is by no means a consequence occurring in all cases of activation of CTLs. For this reason, the following Chromium-Release Assay was additionally performed.

3^{rd} Experiment: Chromium-Release-Assays

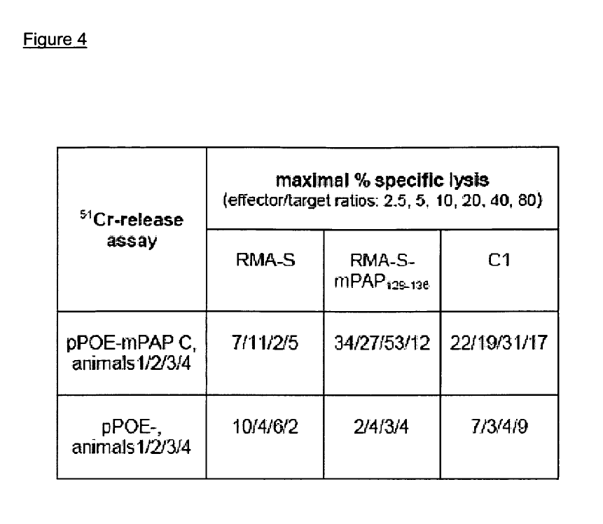

Again, we have immunized BL/6 mice intramuscularly (analogous to the 2^{nd} experiment) with plasmid DNA. Here, we have used the mPAP C gene only which was most successful in the Elispot-Assays (see above in Experiment 2). In chromium-release assays, radioactive (chromium) labeled target cells were co-incubated with splenocytes from with mPAP C-immunized animals. In this assay the activity of cytotoxic cells is determined on the basis of their ability to lyse “target cells” marked with radioactive chromium. Target cells were either unlabeled cells (RMA-S, no PAP antigen onto surface), cells labeled with PAP peptide (RMA-S-mPAP) or PAP-expressing prostate tumor cell line C1. Data gives the percentage of target cell lysis at different ratios of splenocytes/target cells. Here, we have clearly demonstrated, that PAP-immunized animals (mPAPC) induce specific lysis of target cells in vitro whereas controls (empty vector pPOE) did not (see FIG. 4 showing the maximal specific lysis/animal).

4^{th} Experiment: C1-Tumor Regression Experiments

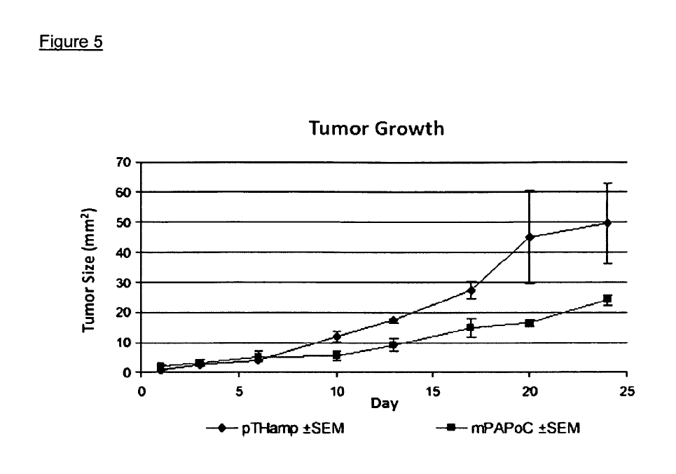

In a first set of tumor regression experiments, PAP-expressing C1 prostate tumor cells (derived from the “TRAMP” mouse, see above and below) were injected subcutaneously in the right shaved flank of male BL/6 mice. When small tumors (2 mm in diameter) were palpable in all animals the first DNA-injection (mPAP C or empty control plasmid) was applied intramuscularly (i.m.) in both musculus tibialis anterior. The boost-vaccinations were performed on days 7 and 14 (FIG. 5). Data show a reduced tumor growth in PAP C treated mice.

5^{th} Experiment: C1-Tumor Regression Experiments

The TRAMP (transgenic adenocarcinoma of the mouse prostate) model represents a system which mimics the natural situation of PCa development [Greenberg, N. M. et al., Proc Natl Acad Sci 92 (1995) 3439-3443]. These mice express the SV40 large T antigen (Tag) under the control of a prostate-specific androgen-dependent rat probasin-promotor leading to prostate cancer in males during development. In this model, PAP is expressed in the thymus in sufficient (low) amounts [Zheng, X. et al., J Immunol 169 (2002) 4761-4769] to enable negative selection of high-avidity T cell clones and is in the periphery selectively expressed under the influence of sexual hormones [Greenberg, N. M. et al., Proc Natl Acad Sci 92 (1995) 3439-3443]. During puberty (after week 4) animals progressively develop intraepithelial prostate neoplasia resulting in a progression to invasive carcinoma of epithelial origin [Shappel, S. B. et al., Cancer Res 64 (2004) 2270-2305] and consequently metastasis [Huss, W. J. et al., Semin Cancer Biol 11 (2001) 245-260], very similar to the human pathology [DeMarzo, A. M. et al., Lancet 361 (2003) 955-9641].

It was shown, that TRAMP mice characteristically express the large T antigen by 8 weeks of age. By 10 weeks of age, animals develop a distinct pathology in the epithelium of the dorsolateral prostate and only two weeks later (week 12) distant site metastasis can be detected (commonly in periaortic lymph nodes and lungs) [Gingrich, J. R. et al., Cancer Research 56 (1996) 4096-4102].

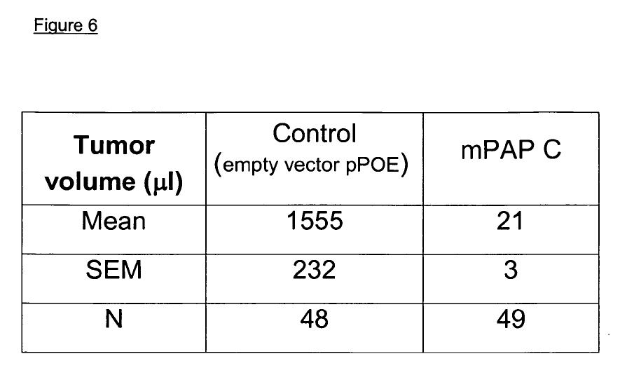

In this set of experiments we have immunized TRAMP animals in weeks 10, 12 and 14 with the mPAP C gene (or empty control plasmid) intramuscularly in both musculus tibialis anterior, respectively (FIG. 6). Tumor volumes were measured by magnetic resonance imaging. Here, It has been very clearly demonstrated that PAP C vaccination prevents outgrowth of prostate cancer in the TRAMP model.

1. A preventive or therapeutic agent for the prevention or treatment of prostate cancer, wherein said agent comprises a recombinant Prostate Acid Phosphatase (PAP) nucleic acid or a functional equivalent thereof, and wherein said agent is in the form of a fusion polynucleotide comprising
a deletion of the signal sequence,
codon optimization for humans,
linkage with an SV 40 enhancer,
linkage with a J-domain, and
linkage with a Kozak sequence.

2. The preventive or therapeutic agent according to claim 1, wherein the functional equivalent shows a homology of at least 70% to mouse PAP DNA, as represented by SEQ ID No: 3.

3. The preventive or therapeutic agent according to claim 1, wherein said agent is a recombinant DNA and a functional equivalent, wherein said functional equivalent comprises an epitope or a minigene of a PAP nucleic acid.

4. The preventive or therapeutic agent according to claim 1, wherein said agent is selected from the group consisting of mPAP A, mPAP B and mPAP C.

5. A method for the prevention or treatment of prostate cancer, comprising administering to a subject an agent comprising a recombinant Prostate Acid Phosphatase (PAP) nucleic acid or a functional equivalent thereof wherein said agent is in the form of a fusion polynucleotide comprising
a deletion of the signal sequence,
codon optimization for humans,
linkage with an SV 40 enhancer,
linkage with a J-domain, and
linkage with a Kozak sequence.

6. The method according to claim 5 wherein the functional equivalent shows a homology of at least 70% to mouse PAP DNA, as represented by SEQ ID No: 3.

7. The method according to claim 5, wherein said agent is a recombinant DNA, and a functional equivalent, wherein said functional equivalent comprises an epitope or a minigene of a PAP nucleic acid.

8. The method according to claim 5, wherein said agent is selected from the group consisting of mPAP A, mPAP B and mPAP C.

9. The method according to claim 5, wherein the treatment or prevention of prostate cancer is accompanied by or follows a treatment with further conventional therapy.

10. Vector, comprising the nucleic acid according to claim 1.

11. Host cell, comprising the vector according to claim 10.

12. Method for the production of a nucleic acid according to claim 1, comprising the following steps:
a) providing a recombinant DNA comprising a PAP DNA or a functional equivalent thereof, wherein at least all introns have been deleted and
b) deleting the signal sequence and
c) codon-optimizing the resultant recombinant DNA and
d) linking the PAP DNA or functional equivalent with an SV enhancer, and with a Kozak sequence, and
e) expressing the resultant construct.

13. The preventive or therapeutic agent according to claim 1, wherein the functional equivalent shows a homology of at least 80% to mouse PAP DNA, as represented by SEQ ID No: 3.

14. The preventive or therapeutic agent according to claim 1, wherein the functional equivalent shows a homology of at least 90% to mouse PAP DNA, as represented by SEQ ID No: 3.

15. The method according to claim 5 wherein the functional equivalent shows a homology of at least 70% to mouse PAP DNA, as represented by SEQ ID No: 3.

16. The method according to claim 5 wherein the functional equivalent shows a homology of at least 80% to mouse PAP DNA, as represented by SEQ ID No: 3.

17. The method according to claim 5 wherein the functional equivalent shows a homology of at least 90% to mouse PAP DNA, as represented by SEQ ID No: 3.
